(19) United States
(12) Patent Application Publication
(10) Pub. No.: US 2023/0104658 A1
(43) Pub. Date: Apr. 6, 2023

(54) METHODS OF INCREASING PROGENITOR CELL PRODUCTION

(71) Applicants: Janssen Pharmaceutica NV, Beerse (BE); Montefiore Medical Center, Bronx, NY (US)

(72) Inventors: Gary EICHENBAUM, Belle Mead, NJ (US); Chandan Guha, Scarsdale, NY (US); Justin Vercellino, Bronx, NY (US)

(21) Appl. No.: 17/937,155

(22) Filed: Sep. 30, 2022

Related U.S. Application Data
(60) Provisional application No. 63/261,957, filed on Oct. 1, 2021.

(51) Int. Cl. A61K 38/19; C12N 5/0789; C12N 5/071; A61K 47/10; A61K 31/395; A61K 9/00; A61K 35/12; A01N 1/02

(52) U.S. Cl. CPC A61K 38/196; C12N 5/0647; C12N 5/069; A61K 47/10; A61K 31/395; A61K 9/0019; A61K 35/12; A01N 1/02; C12N 2501/145; C12N 2501/26

(57) ABSTRACT

Methods of increasing progenitor cell production are described. In particular, an effective amount of a thrombopoietin (TPO) mimetic is used to increase production of at least one cell selected from a stem cell, a progenitor cell or an endothelial cell in the bone marrow of non-irradiated subjects.

### CROSS-REFERENCE TO RELATED APPLICATIONS

This application claims priority to U.S. Provisional Application No. 63/261,957, filed Oct. 1, 2021, the contents of which are incorporated by reference herein in its entirety.

### REFERENCE TO SEQUENCE LISTING SUBMITTED ELECTRONICALLY

This application contains a sequence listing, which is submitted electronically via USPTO Patent Center as an XML formatted sequence listing with a file name “004852-186US1 sequence listing.txt”, creation date of Sep. 29, 2022, and having a size of about 8 kb. The sequence listing submitted via USPTO Patent Center is part of the specification and is herein incorporated by reference in its entirety.

### BACKGROUND

The bone marrow contains various types of progenitor cells, including hematopoietic progenitor cells, fibrocytes, mesenchymal stem cells, and endothelial progenitor cells (EPCs). When an organ or tissue is injured, progenitor cells from the bone marrow travel through the circulation system and reach the injured site for tissue repair and regeneration (Rennert et al., Regen Med, 7:833-50 (2012)). For example, it has been demonstrated that EPCs recruited into ischemic tissues can stimulate angiogenesis and lead to tissue repair and regeneration (Yoder et al., Blood, 109:1801-1809 (2007) and Nolan et al., Genes Dev, 21:1546-1558 (2007)).

Increasing stem and progenitor cell production is also critical for adoptive cell transfer, which is the infusion into patients of autologous or allogeneic cells of various hematopoietic lineages to treat disease. The use of cells from peripheral blood is preferred for most autologous transplantations and a significant proportion of allogeneic transplantations because of higher stem cell and progenitor cell content as compared to bone marrow or cord blood. The minimum threshold for autologous transplantation of peripheral blood stem cells is >2×10⁶ CD34/kg, which may not always be achieved using optimal doses of granulocyte-colony stimulating factor (G-CSF).

Thrombopoietin (TPO) is the primary physiological regulator of platelet production. It is known that TPO regulates platelet levels by binding to c-MPL on megakaryocytes (to stimulate platelet maturation) and existing platelets (providing negative feedback) (Mitchell and Bussell, Semin. Hematol. 52(1):46-52 (2015)). In addition to its regulation of platelet function, TPO has demonstrated a role in maintaining hematopoietic stem cell viability following radiation and chemotherapy (Mouthon et al., Int J Radiat Oncol Biol Phys, 43:867-875 (1999)), preventing apoptosis of irradiated bone marrow cells (Drouet et al., 1999), causing expansion of the stem cell population in combination with other cytokines (Ramsfj ell et al., J Immunol, 158:5169-5177 (1997)), enhancing in vivo platelet and erythroid recovery following irradiation (Neelis et al., Blood, 92:1586-1597 (1998)), and enhancing stem cell mobilization into peripheral blood (Torii et al., Br J Haematol, 103:1172-1180 (1998)). However, recombinant human TPO (rhTPO) is not a viable therapy in humans, due to induction of cross-reactive antibodies to endogenous TPO that can lead to chronic thrombocytopenia (Li et al., Blood 98(12):3241-8 (2001)).

### BRIEF SUMMARY

The application relates to methods of increasing production of at least one cell selected from the group consisting of a stem cell, a progenitor cell and an endothelial cell in a non-irradiated subject. In one general aspect, the application relates to a method of increasing production of at least one of a hematopoietic progenitor cell, a myeloid progenitor cell, an endothelial progenitor cell and an endothelial cell in a non-irradiated subject, the method comprising administering to the subject an effective amount of a thrombopoietin (TPO) mimetic comprising the amino acid sequence of SEQ ID NO: 1, preferably having the following structure of formula (I), or a pharmaceutically acceptable salt or ester thereof:

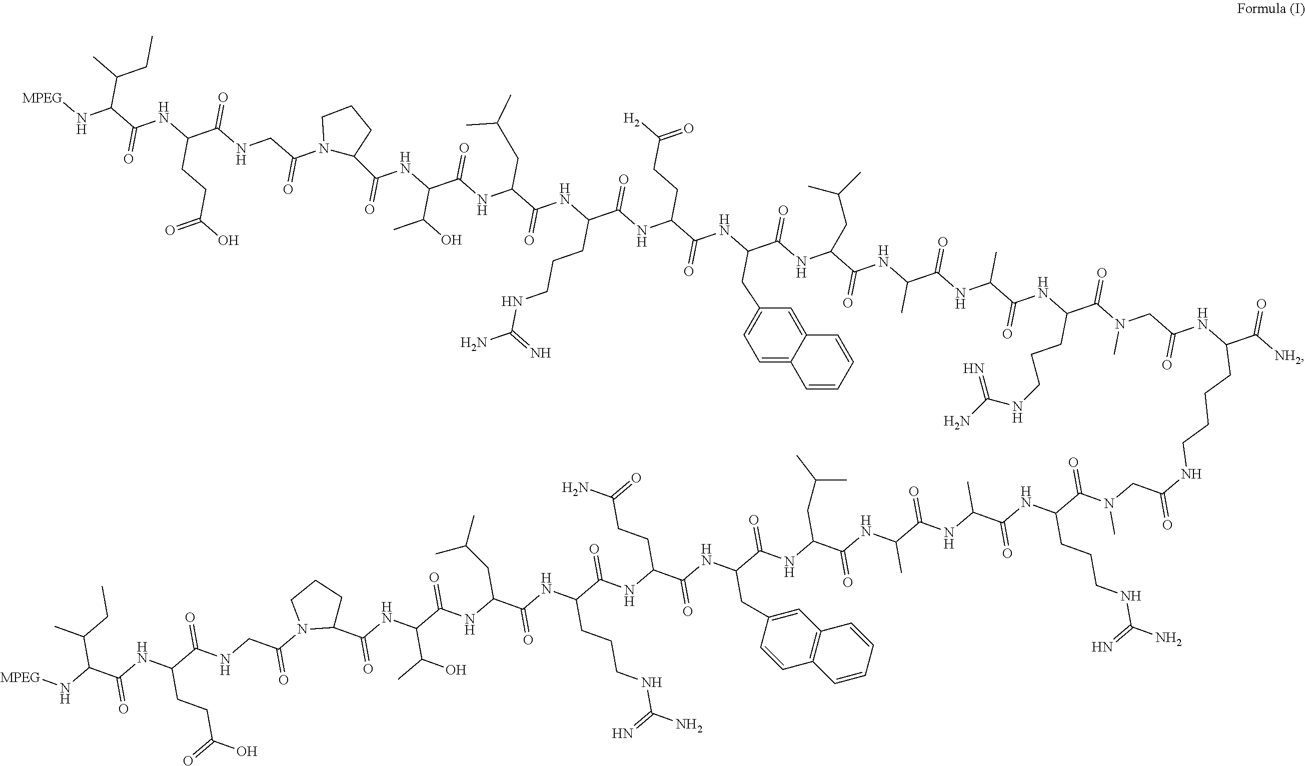

wherein MPEG represents methoxypolyethyleneglyco120000; or having the amino acid sequence of SEQ ID NO: 3 (romiplostim).

Also provided is a method of obtaining at least one cell selected from the group consisting of a hematopoietic progenitor cell, a myeloid progenitor cell, an endothelial progenitor cell and an endothelial cell from a non-irradiated subject, comprising: (a) administering an effective amount of a thrombopoietin (TPO) mimetic to the subject to increase the at least one cell in the subject; (b) harvesting the at least one cell from the subject 7 to 21 days after the administering; and optionally (c) isolating the at least one cell; wherein the TPO mimetic comprises the amino acid sequence of SEQ ID NO: 1, preferably the TPO mimetic has the following structure of formula (I), or a pharmaceutically acceptable salt or ester thereof:

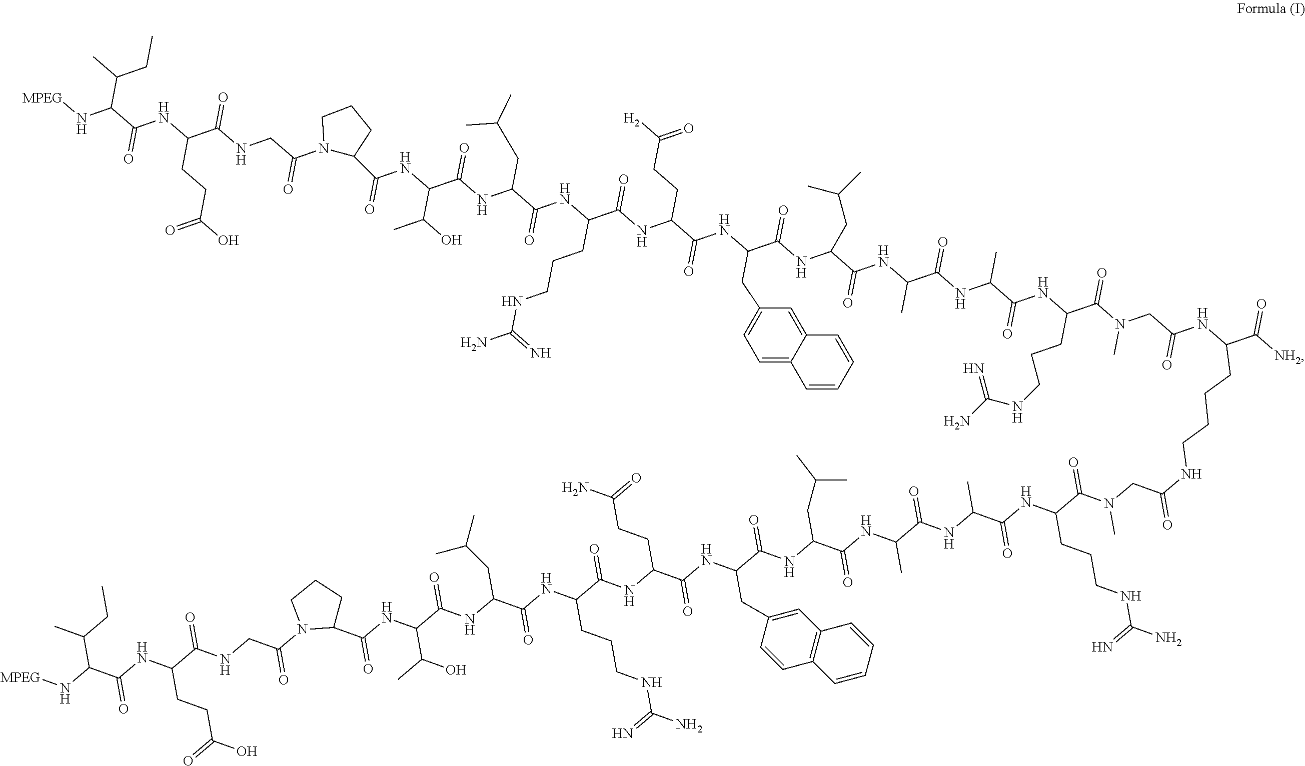

wherein MPEG represents methoxypolyethyleneglyco120000; or has the amino acid sequence of SEQ ID NO: 3 (romiplostim).

Also provided is a method of providing at least one cell selected from the group the group consisting of a hematopoietic progenitor cell, a myeloid progenitor cell, an endothelial progenitor cell and an endothelial cell to a subject, comprising: (a) administering an effective amount of a thrombopoietin (TPO) mimetic to the subject to increase production of the at least one cell in the subject; (b) harvesting the at least one cell from the subject 7-21 days after the administering; (c) optionally isolating the at least one cell; and (d) administering the at least one cell or the isolated at least one cell to the subject.

In certain embodiments, the method further comprises administering to the subject a mobilizing agent prior to harvesting the at least one cell. In certain embodiments, the mobilizing agent is administered after the TPO mimetic is administered. In preferred embodiments, the mobilizing agent is plerixafor or an FLT-3 ligand.

Also provided is a method of treating a disease or disorder in a subject in need thereof, comprising administering to the subject an effective amount of at least one cell selected from the group consisting of a hematopoietic progenitor cell, a myeloid progenitor cell, an endothelial progenitor cell and an endothelial cell to the subject, wherein the at least one cell is obtained by a method comprising: (a) administering an effective amount of a thrombopoietin (TPO) mimetic to the subject to increase production of the at least one cell in the subject; and (b) harvesting the at least one cell.

In certain embodiments, the method further comprises administering plerixafor to the subject 7-20 days after administering the TPO mimetic. For instance, the plerixafor may be administered 7 days, 8 days, 9 days, 10 days, 11 days, 12 days, 13 days, 14 days, 15 days, 16 days, 17 days, 18 days, 19 days, or 20 days after administering the TPO mimetic.

In certain embodiments, the method further comprises treating the subject with a bone marrow ablative treatment after harvesting the at least one cell.

In certain embodiments, the subject is in need of treatment for a disease selected from the group consisting of cancer, peripheral vascular insufficiency, stroke, cardiovascular injury, lung injury, liver injury and kidney injury.

In certain embodiments, the number of at least one cell is increased within the subject's bone marrow. In certain embodiments, the number of at least one cell is increased within the subject's peripheral circulation.

In certain embodiments, the at least one cell is harvested from the subject's peripheral circulation, and optionally isolated.

In certain embodiments, the at least one cell is cryopreserved after isolation. In other embodiment, the at least one cell is cryopreserved before isolation.

In certain embodiments, the cryopreserved at least one cell is thawed and determined to be viable prior to administering the cells to the subject.

In certain embodiments, the at least one cell is administered to the subject when the subject is in need of such cell administration.

In certain embodiments, the subject is a human.

In certain embodiments, the TPO mimetic is administered to the subject by intravenous, intramuscular, intracutaneous, or subcutaneous injection.

In certain embodiments, the subject is administered a single dose of the effective amount of the TPO mimetic. In other embodiments, the subject is administered more than one dose of the effective amount of the TPO mimetic.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings. As used in FIGS. 1 to 6, “TPOm” refers to JNJ-26366821 and RWJ-800088.

FIGS. 1A-1B show changes in megakaryocyte populations in mouse bone marrow with TPOm treatment over the time. FIG. 1A shows representative Hematoxylin and Eosin (H&E) images of sternal bone marrow from naive and D6 TPOm-treated mice. Scale bar is 100 pm. FIG. 1B shows count of megakaryocytes in the sternal bone marrow of naive and TPOm-treated mice at the indicated day post-injection (n=3/group).

FIG. 2 shows the gating strategy for identifying various hematopoietic stem cell populations as represented by naive and D6 TPOm-treated mice.

FIGS. 3A-3D show changes in hematopoietic cell populations in mouse bone marrow with

TPOm treatment over the time. FIG. 3A shows count of myeloid progenitor cells (MPCs) per femur of naive and TPOm-treated mice (n=4/group). FIG. 3B shows count of the lineage-, c-kit+, Sca-1+(LSK) per femur of naive and TPOm-treated mice (n=4/group). FIG. 3C shows count of short-term (ST-) hematopoietic stem cells (HSCs) per femur of naive and TPOm-treated mice (n=4/group). FIG. 3D shows count of long-term (LT-) HSCs per femur of naive and TPOm-treated mice (n=4/group).

FIGS. 4A-4C show changes in endothelial cell populations in mouse bone marrow with TPOm treatment over the time. FIG. 4A shows the gating strategy for identifying various endothelial cell populations as represented by naive and D6 TPOm-treated mice. FIG. 4B shows the number of endothelial progenitor cells (EPC) per femur of naive and TPOm-treated mice (n=4/group). FIG. 4C shows the number of CD31+endothelial cells (EC) per femur of naive and TPOm-treated mice (n=4/group).

FIGS. 5A-5D show the effect of TPOm, treatment and combination TPOm and plerixafor treatment on cell populations in mouse bone marrow after ten days. FIG. 5A shows the percentage of CD45-cells that were identified as myeloid progenitor cells (MPCs) in naive, plerixafor-treated, TPOm-treated, and TPOm+plerixafor-treated mice (n=3/group). FIG. 5B shows the percentage of CD45-cells that were identified as hematopoietic stem cells (HSCs) in naive, plerixafor-treated, TPOm-treated, and TPOm and plerixafor-treated mice (n=3/group). FIG. 5C shows the percentage of CD45-cells that were identified as endothelial cells (ECs) in naive, plerixafor-treated, TPOm-treated, and TPOm and plerixafor-treated mice (n=3/group). FIG. 5D shows the percentage of CD45-cells that were identified as endothelial progenitor cells (EPCs) in naive, plerixafor-treated, TPOm-treated, and TPOm and plerixafor-treated mice (n=3/group).

FIGS. 6A-6B show the effect of plerixafor and TPOm treatment on endothelial cell populations in the peripheral blood of mice. FIG. 6A shows the percentage of CD45-cells identified as circulating endothelial cells (CECs) in naive, plerixafor-treated, TPOm-treated, and TPOm and plerixafor-treated mice (n=3/group). FIG. 6B shows the percentage of CD45-cells identified as endothelial progenitor cells (EPCs) in naive, plerixafor-treated, TPOm-treated, and TPOm and plerixafor-treated mice (n=3/group).

### DETAILED DESCRIPTION

This disclosure is based upon, at least in part, on the identification of a thrombopoietin (TPO) mimetic as a therapeutic for increasing stem cell and/or progenitor cell production in the bone marrow of non-irradiated subjects.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the application. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the application described herein. Such equivalents are intended to be encompassed by the application.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human, to whom will be or has been vaccinated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, the term “in combination”, in the context of the administration of two or more therapies to a subject, refers to the use of more than one therapy. The use of the term “in combination” does not restrict the order in which therapies are administered to a subject.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

TPO mimetic

As used herein, a “TPOm”, “TPO mimetic” or “thrombopoietin mimetic” refers to a compound comprising a peptide capable of binding to and activating a thrombopoietin receptor. Preferably, in a TPO mimetic useful for the methods of the application, the peptide capable of binding to and activating a thrombopoietin receptor has no significant homology with thrombopoietin (TPO). The lack of homology with TPO reduces the potential for generation of TPO antibodies. Examples of such peptide useful in a TPO mimetic include, but are not limited to, those described in U.S. Publication Nos. 2003/0158116; 2005/0137133; 2006/0040866; 2006/0210542; 2007/0148091; 2008/0119384; U.S. Pat. Nos. 5,869,451; 7,091,311; 7,615,533; 8,227,422; International Patent Publications WO2007/021572; WO2007/094781; and

WO2009/148954, the entire contents of each of which are incorporated herein by reference. More preferably, in a TPO mimetic useful for the methods of the application, the peptide capable of binding to and activating a thrombopoietin receptor is covalently linked to a moiety that improves one or more properties of the peptide. By way of a non-limiting example, the moiety can be a hydrophilic polymer, including but not limited to polyethylene glycol (PEG), polypropylene glycol, polylactic acid and polyglycolic acid. The moiety can also be a polypeptide, such as a Fc region or an albumin.

In a preferred embodiment, a TPO mimetic useful for the methods of the application comprises a peptide having the amino acid sequence of: IEGPTLRQXaaLAARYaa (SEQ ID NO:1), wherein Xaa is tryptophan (W) or P-(2-naphthyl)alanine (referred to herein as “2-Nal”), and Yaa is alanine (A) or sarcosine (referred herein as “Sar”). Preferably, the peptide of SEQ ID NO:1 is covalently linked to a PEG or fused to a Fc domain.

In some embodiments, a TPO mimetic useful for the methods of the application comprises a peptide of SEQ ID NO:1 covalently linked to a PEG, preferably a PEG having an average molecular weight of between about 5,000 to about 30,000 daltons. Preferably, the PEG is selected from the group consisting of monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM). The PEGylation of the peptide leads to a reduced clearance of the compound without loss of potency. See, e.g., U.S. Pat. No. 7,576,056, the entire contents of which are incorporated herein by reference.

In one preferred embodiment, a TPO mimetic useful for the methods of the application is RWJ-800088 also referred to as JNJ-26366821 or a derivative thereof. As used herein, “RWJ-800088” refers to a 29-mer peptide having two identical 14-mers linked by a lysinamide residue as follows:

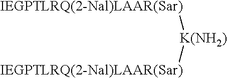

and having a methoxypoly(ethylene glycol) (MPEG) covalently linked to each N-terminal isoleucine, or a pharmaceutically acceptable salt or ester thereof. The 14-mers is identical to SEQ ID NO:1, wherein Xaa is 2-Nal and Yaa is Sar, The RWJ-800088 is thus composed of two 14 amino acid peptide chains of SEQ ID NO:2 (IEGPTLRQ(2-Nal)LAAR(Sar)) linked by lysinamide reside, and each N-terminal isoleucine is linked to a methoxy polyethylene glycol (MPEG) chain. Accordingly, RWJ-800088 has an abbreviated molecular structure of (MPEG-Ile-Glu-Gly-Pro-Thr-Leu-Arg-Gln-(2-Nal)-Leu-Ala-Ala-Arg-(Sar))2-Lys-NH2; wherein (2-Nal) is Beta-(2-naphthyl)alanine, (Sar) is sarcosine and MPEG is methoxypoly(ethylene glycol), or a pharmaceutically acceptable salt or ester thereof. Preferably, the MPEG has an approximately 20,000 Dalton molecular weight or represents methoxypolyethylene glyco120000.

In one embodiment, RWJ-800088 has a molecular structure of formula (I), or a pharmaceutically acceptable salt or ester thereof:

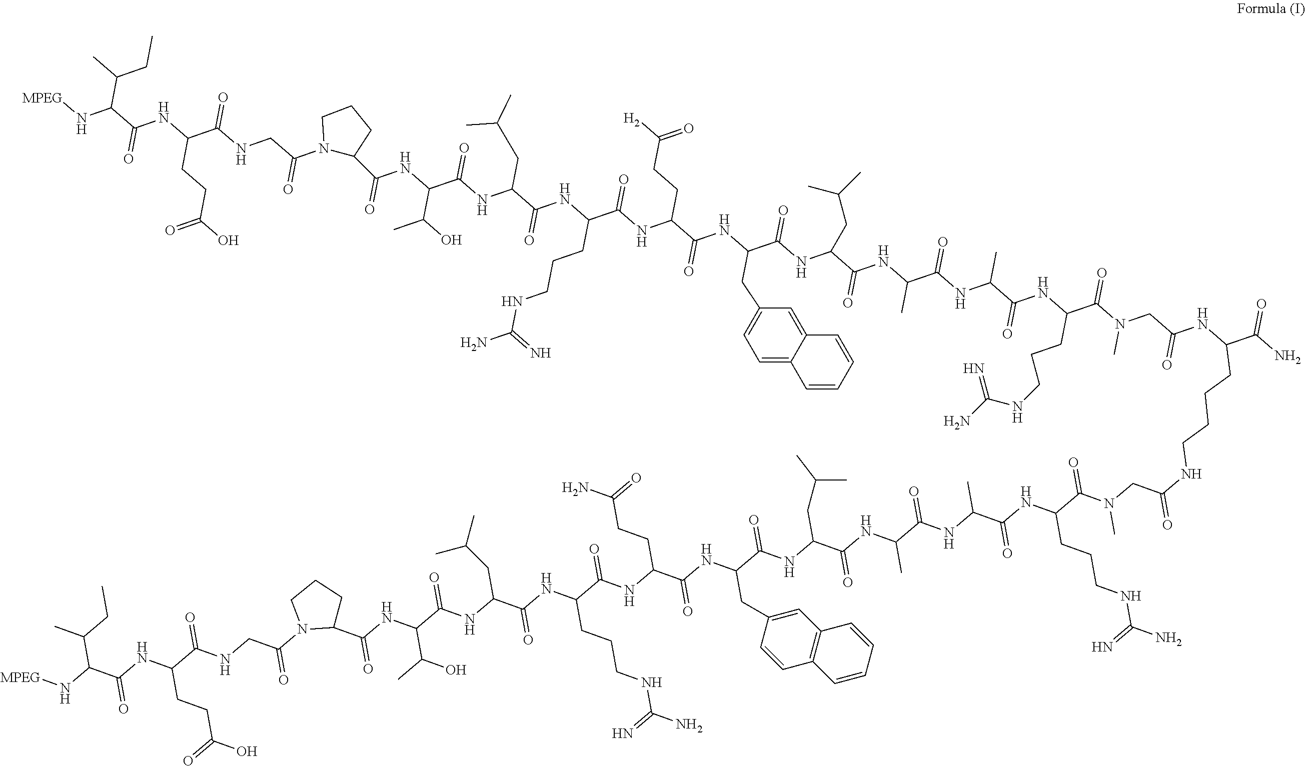

In a preferred embodiment, the MPEG in RWJ-800088 is methoxypolyethyleneglyco120000, and the RWJ-800088 has the full chemical name of: methoxypolyethyleneglyco120000-propionyl-L-Isoleucyl-L-Glutamyl-Glycyl-L-Prolyl-L-Threonyl-L-Leucyl-L-Arginyl-L-Glutaminyl-L-2-Naphthylalanyl-L-Leucyl-L-Alanyl-L-Alanyl-L-Arginyl-Sarcosyl-Ne-(methoxypolyethyleneglyco120000-propionyl-L-Isoleucyl-L-Glutamyl-Glycyl-L-Prolyl-L-Threonyl-L-Leucyl-L-Arginyl-L-Glutaminyl-L-2-Naphthylalanyl-L-Leucyl-L-Alanyl-L-Alanyl-L-Arginyl-Sarcosyl-)-Lysinamide, or a pharmaceutically acceptable salt or ester thereof. The molecular weight of the peptide without PEG is 3,295 Daltons and with two 20,000 Dalton MPEG chains is approximately 43,295 Daltons.

In some embodiments, a TPO mimetic useful for the methods of the application comprises a peptide of SEQ ID NO:1 fused to a Fc domain. Fusing the peptide to a Fc domain can stabilize the peptide in vivo. See, e.g., U.S. Pat. No. 6,660,843, the entire contents of which are incorporated herein by reference.

In another preferred embodiment, a TPO mimetic useful for the invention is romiplostim. As used herein, “romiplostim” refers to fusion protein having a Fc domain linked to the N-terminal isoleucine of the peptide of SEQ ID NO:1, where Xaa is W and Yaa is A. In particular, romiplostim has the following amino acid sequence:

(SEQ ID NO: 4)
MDKTHTCPPCPAPELLGGPSVFLFPPKPKDTLMISRTPEVTCVVVD
VSHEDPEVKFNWYVDGVEVHNAKTKPREEQYNSTYRVVSVLTVLHQ
DWLNGKEYKCKVSNKALPAPIEKTISKAKGQPREPQVYTLPPSRDE
LTKNQVSLTCLVKGFYPSDIAVEWESNGQPENNYKTTPPVLDSDGS
FFLYSKLTVDKSRWQQGNVFSCSVMHEALHNHYTQKSLSLSPGKGG
GGGIEGPTLRQWLAARAGGGGGGGGIEGPTLRQWLAARA,

It has the thrombopoietin receptor binding domain amino acid sequence of IEGPTLRQWLAARA (SEQ ID NO:3).

### Method of Increasing Progenitor Cell Production

The TPO mimetics described above, e.g., a TPO mimetic comprising SEQ ID NO: 1, e.g., RWJ-800088 having a structure of formula (I), or romiplostim comprising the amino acid sequence of SEQ ID NO: 3, preferably having the amino acid sequence of SEQ ID NO: 4, can be used in the methods of the application described herein. The methods of the application relate to increasing progenitor cell and/or endothelial cell production in non-irradiated subjects. The methods of administering an effective amount of a thrombopoietin (TPO) mimetic are effective to increase cell populations within the subject's bone marrow and peripheral circulation.

The application relates to a method of increasing production of at least one cell selected from the group consisting of a stem cell, a progenitor cell and an endothelial cell in a non-irradiated subject, the method comprising administering to the subject an effective amount of a thrombopoietin (TPO) mimetic, such as a TPO mimetic comprising the amino acid sequence of SEQ ID NO: 1, preferably a TPOm having the following structure of formula (I), or a pharmaceutically acceptable salt or ester thereof:

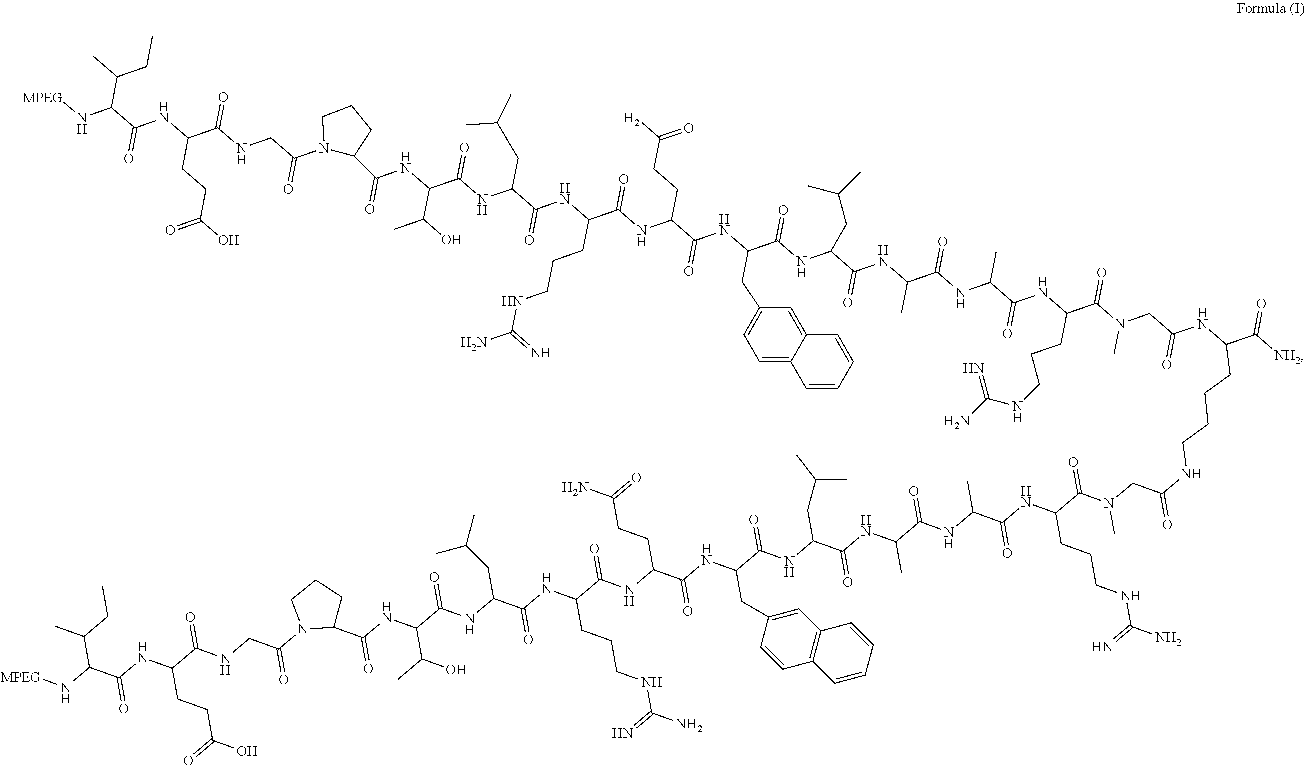

wherein MPEG represents methoxypolyethyleneglyco120000; or romiplostim comprising the amino acid sequence of SEQ ID NO: 3, preferably having the amino acid sequence of SEQ ID NO: 4.

In certain embodiments, the application relates to a method of increasing production of a stem cell. The term “stem cell” as used herein refers to undifferentiated cells of a multicellular organism having the ability to self-renew that can generate daughter cells that can undergo terminal differentiation into more than one distinct cell types with specific functions. A stem cell has the potential to differentiate into multiple types of cells and is capable of unlimited self-replication via asymmetric cell division, a process known as self-renewal. In preferred embodiment, the stem cell is an adult stem cell, also called a somatic stem cell, which is multipotent and can generate cell types within a specific lineage, such as blood cells or endothelial cells.

In certain embodiments, the stem cell is a hematopoietic stem cell. As used herein, the term “hematopoietic stem cells” (“HSCs”) refers to immature cells having the capacity to self-renew and to differentiate into one or more mature blood cells. Examples of mature blood cells include, but are not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). In certain embodiments, a hematopoietic stem cell is characterized as Lineage-, c-kit+, Sca-1+, CD34+. In other embodiments, a hematopoietic stem cell is characterized as Lineage-, c-kit+, Sca-1+, CD34-, CD48-, CD150+.

In certain embodiments, the application relates to a method of increasing production of a progenitor cell. The term “progenitor cell” as used herein refers to cells that are descendants of stem cells, which can further differentiate to create specialized cell types. The term is not limitative and does not limit these cells to a particular lineage. Unlike stem cells, progenitor cells have a lesser ability to self-renew. In addition, the cell potency of progenitor cells is usually more restricted than stem cells. A progenitor cell is normally only capable of differentiating into cells that belong to the same tissue or organ. Some progenitor cells have one final target cell that they differentiate to (unipotent), while others have the potential to terminate in more than one cell type (oligopotent or multipotent).

In certain embodiments, the progenitor cell is selected from the group consisting of a hematopoietic progenitor cell (HPC), a myeloid progenitor cell (MPC), and an endothelial progenitor cell (EPC).

The HPCs are immature cells developed from HSCs, which can further differentiate into MPCs or lymphoid progenitor cells (LPC). The MPCs can further differentiate into one of the following mature blood cells: red blood cells/erythrocytes, platelets, mast cells, osteoclasts, granulocytes ((polymorphonuclear leukocytes [PMNs]: neutrophils, eosinophils, and ba sop 5), monocyte-macrophages and dendritic cells. The LPCs, also known as lymphoblasts, are precursors, to other mature blood cell types, including: T-cells/T-lymphocytes, B-cells/B-lymphocytes, NK-cells/Natural killer cells.

The EPCs are circulating cells that express a variety of cell surface markers similar to those expressed by vascular endothelial cells, adhere to endothelium at sites of hypoxia/ischemia, and participate in new vessel formation. Circulating endothelial progenitor cells (EPCs) are bone marrow -derived mononuclear cells that have the capacity to migrate, proliferate, and differentiate into mature endothelial cells (ECs). Circulating EPC cell number and function are reduced in subjects with cardiovascular risk factors and more severe cardiovascular disease (Cubbon et al., Clin Sci (Lond) 2009; 117:173-190; Vasa et al., Circ Res. 2001;89:E1—E7).

In certain embodiments, the method of the application results in an increase in endothelial cell production. The term “endothelial cell” as used herein refers to cells that can form the barrier between vessels and tissues. They are the main type of cells found in the inside lining of blood vessels, lymph vessels, and the heart. In some embodiments, the ECs are circulating ECs in peripheral blood.

In certain embodiment, a method of the application results in an increased production of at least two of a stem cell, a progenitor cell and an endothelial cell.

In another embodiment, a method of the application results in an increased production of a stem cell, a progenitor cell and an endothelial cell.

In certain embodiments, the at least one of stem cells, progenitor cells, and endothelial cells are increased within the subject's bone marrow. In other embodiments, the at least one of stem cells, progenitor cells and endothelial cells are increased within the subject's peripheral circulation.

The application also relates to a method of obtaining at least one of a hematopoietic progenitor cell, a myeloid progenitor cell, an endothelial progenitor cell and an endothelial cell from a subject, comprising: (a) administering an effective amount of a thrombopoietin (TPO) mimetic, such as a TPO mimetic comprising the amino acid sequence of SEQ ID NO: 1, preferably RWJ-800088 having the structure of formula (I), or romiplostim comprising the amino acid sequence of SEQ ID NO: 3, preferably having the amino acid sequence of SEQ ID NO: 4, to the subject to increase the at least one cell in the subject; (b) harvesting the at least one cell, preferably 3 to 21 days, after the administering; and (c) optionally isolating the at least one cell.

The application also provides a method of providing at least one cell selected from the group consisting of a hematopoietic progenitor cell, a myeloid progenitor cell, an endothelial progenitor cell and an endothelial cell to a subject, comprising (a) administering an effective amount of a thrombopoietin (TPO) mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, to the subject to increase production of the at least one cell in the subject; (b) harvesting the at least one cell from the subject 7-21 days after the administering; (c) optionally isolating the at least one cell; and (d) administering the isolated at least one cell to the subject.

The application further provides a method of treating a subject in need thereof, comprising administering to the subject an effective amount of at least one cell selected from the group consisting of a hematopoietic progenitor cell, a myeloid progenitor cell, an endothelial progenitor cell and an endothelial cell, wherein the at least one cell is obtained by a method comprising: a) administering an effective amount of a thrombopoietin (TPO) mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4 to the subject to increase production of the at least one cell in the subject; and (b) harvesting the at least one cell from the subject 7-21 days after the administering. In some embodiments, the harvested at least one cell can be administered to the subject without being isolated. Preferably, the harvested at least one cell is isolated before being administered to the subject. In some embodiments, the at least one cell is isolated, cryopreserved, and thawed before being administered to the subject. In other embodiments, the at least one cell is isolated and administered to the subject without being cryopreserved and thawed.

Certain aspects of the present invention relate to methods of mobilizing stem cells and/or progenitor cells. As used herein, “mobilizing” and “mobilizing stem cells and/or progenitor cells” are used interchangeably to refer to the act of inducing the migration of the stem cells and/or progenitor cells from a first location (e.g., stem cell niche, e.g., bone marrow) into a second location (e.g., tissue (e.g., peripheral blood) or organ (e.g., spleen).

In certain embodiments, the method further comprises administering to the subject a mobilizing agent prior to harvesting the one or more stem and/or progenitor cells. Preferably, the mobilizing agent is administered to the subject after the TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, is administered. In some embodiments, the mobilizing agent, e.g., plerixafor, or an FLT-3 ligand, is administered to the subject with the TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4. In certain embodiments, the mobilizing agent is selected from the group consisting of granulocyte-colony stimulating factor (G-CSF), glycosylated G-CSF, pegylated G-CSF, granulocyte macrophage colony stimulating factor (GM-CSF), CXCR4 antagonists (e.g., plerixafor), CXCR4 inhibitors (e.g., POL6326, BKT140, TG-0054 and NOX-Al2), VLA4 antagonists (e.g., anti-VLA4 antibodies such as natalizumab), VCAM-1 inhibitors, CD44 antagonists, integrin a4f3 1 antagonists (e.g., BI05192), proteasome inhibitors (e.g., bortezomib), parathormone (PTH), CXCL2 (GroP), cyclophosphamide, 5-fluorouracil, cisplatin, etoposide, ifosfamide, cytarabine, fms like tyrosine kinase 3 ligand (FLT-3 ligand), and combinations thereof; preferably, the mobilizing agent is plerixafor or an FLT-3 ligand. Plerixafor (AMD3100) is well known in the art and disclosed in U.S. Pat. Nos. 5,583,131; 6,987,102; and 7,897,590; the contents of winch are incorporated by reference in entirety. FLT-3 ligand has been demonstrated to cooperate with TPO to generate dendritic precursors from human hematopoietic progenitors, as described in Chen, Wei et al. “Thrombopoietin cooperates with FLT3-ligand in the generation of plasmacytoid dendritic cell precursors from human hematopoietic progenitors.” Blood vol. 103,7 (2004): 2547-53, which is herein incorporated by reference in its entirety.

In certain embodiments, the cells are harvested from the subject's peripheral circulation. In certain embodiments, the cells are harvested from the subject's peripheral blood 3 to 21 days, such as 3, 4, 5, 6, 7, 8, 9, 10, 11, 12, 13, 14, 15, 16, 17, 18, 19 or 21 days, after the administration of the effective amount of the TPO mimetic. In certain embodiments, the cells are EPCs, MPCs, and/or circulating ECs, and they are harvested from the subject's peripheral blood 3-21 days, preferably 5-15 days, such as 5, 6, 7, 8, 9, 10, 11, 12, 13, 14, 15 days after the administration of the effective amount of the TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4.

Methods of harvesting cells from the peripheral circulation are well known to one of skill in the art, including, but not limited to, apheresis.

Cells can be isolated by any method known to one of skill in the art in view of the present disclosure, for example, based on expression/lack of expression of certain markers, rates of proliferation, differentiation potential, and/or other properties. In some embodiments, the cells are isolated based on the presence of a particular marker or combination of markers including, for example, CD34, Scal, c-kit, CD31, CD144, and VEGFR2. In some embodiments, the cells are isolated based on the absence of a particular marker, for example, CD45. In other embodiments, negative selection is performed for markers of, for example, T cells, B cells, granulocytic, and/or myelomonocytic cells.

In certain embodiments, the at least one cell is isolated by flow cytometry. In certain embodiments, myeloid progenitor cells are characterized as lineage-, c-kit+. In other embodiments, myeloid progenitor cells are characterized as CD45-, c-kit+, Sca-1-. In certain embodiments, endothelial progenitor cells are characterized as CD45-, TER-119-, (Lineage-), CD31+, CD34+, VEGFR2+. In other embodiments, endothelial progenitor cells are characterized as CD45-, CD31+, CD34+, CD144+, VEGFR2+. In certain embodiments, endothelial cells are characterized as CD45-, TER-119-, (Lineage-), CD31+. In other embodiments, endothelial cells are characterized as CD45-, CD31+, CD144+, VEGFR2+.

In certain embodiments, one or more of the cells are cryopreserved after isolating. The term “cryopreserve” or its various grammatical forms as used herein refers to preserving cells for long term storage in a cryoprotectant at a low temperature. The term “cryoprotectant” as used herein refers to an agent that minimizes ice crystal formation in a cell or tissue, when the cell or tissue is cooled to subzero temperatures and results in no substantially damage to the cell or tissue after warming, in comparison to the effect of cooling without cryoprotectant. Any suitable cryoprotectant can be used to cryopreserve the harvested cells using methods known in the art in view of the present disclosure.

In certain embodiments, one or more cryopreserved cells are thawed and determined to be viable prior to administering the cells into the subject.

In certain embodiments, the method further comprises treating the subject with a bone marrow ablative treatment after harvesting one or more a hematopoietic progenitor cell, a myeloid progenitor cell, an endothelial progenitor cell and an endothelial cell.

As used herein, “bone marrow ablative treatment” refers to a treatment using radiation, high-dose chemotherapy or a combination of both to deplete the bone marrow. The term “radiation” or “radiation therapy”, as used herein, refers to a therapy using ionizing radiation to control cell growth. Radiation therapy includes, but is not limited to, targeted radiation, and total body irradiation therapy. Total body irradiation (TBI), also referred as whole-body radiation therapy, is a form of radiation therapy, which involves irradiation of the entire body. TBI is used primarily as part of the preparative regimen for transplantation of hematopoietic stem cell, bone marrow stem cells or peripheral blood progenitor stem cells, in the treatment of hematopoietic diseases. TBI is done to kill any cancer cells that are left in the body and helps make room in the patient's bone marrow for new blood stem cells to grow. TBI also helps prevent the body's immune system from rejecting the transplanted stem cells. Optimal TBI requires individual treatment planning based on systematic dose measurements and CT-localization under treatment conditions, considering tissue inhomogeneities and individual body contours, careful performance of TBI with verification and control and documentation of all relevant treatment parameters. Methods known to those skilled in the art can be used to conduct the TBI in a method of the invention in view of the present disclosure. See, for example, Quast, J Med Phys. 2006, 31(1): 5-12, for a guideline on TBI, the entire content of which is incorporated herein by reference.

The term “chemotherapy,” as used herein, refers to a treatment of a disease that uses one or more chemical substances (chemotherapeutic agents). Chemotherapeutic agent, also referred to as chemotherapeutic compound, refers to any agent that can be used to treat a disease or disorder of a subject. Conventional chemotherapy uses non-specific cytotoxic drugs to inhibit cell division (mitosis). Based on their principal mechanism of action, conventional chemotherapeutics can be broadly subdivided into: 1) alkylating agents; 2) antimetabolites; 3) topoisomerase inhibitors; 4) microtubular poisons; and 5) cytotoxic antibiotics.

Radiochemotherapy (RCTx, RT-CT), also referred to as chemoradiotherapy (CRT, CRTx) and chemoradiation, is the combination of radiotherapy and chemotherapy to treat cancer. Radiochemotherapy can be concurrent (together) or sequential (one after the other).

In certain embodiments, one or more cells, such as one or more of the isolated cells, are administered to the subject when the subject is in need of such cell administration, such as a part of an adoptive cell transfer.

As used herein, “allogeneic transplantation” refers to transplantation of cells deriving from or originating in a donor who is genetically non-identical to the recipient but of the same species as a part of an adoptive cell therapy. “Autologous transplantation” refers to transplantation of cells deriving from or originating in the same subject as a part of an adoptive cell therapy.

Furthermore, the present application generally relates to methods of tissue repair and/or regeneration, such as regeneration of bone marrow, comprising administering an effective amount of a thrombopoietin (TPO) mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, thereby repairing and/or regenerating tissue in the subject. Moreover, the present application generally relates to methods of increasing stem and/or progenitor cell production for adoptive cell transfer, comprising administering an effective amount of a thrombopoietin (TPO) mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, thereby increasing stem and/or progenitor cell production for adoptive cell transfer. In some instances, the subject receives the cells for the adoptive cell transfer from a donor (allogeneic transplantation). In some instances, the subject is treated with the TPO mimetic, and, subsequently, the subject's own cells are later readministered to the subject for the adoptive cell therapy (autologous transplantation).

The present invention contemplates treating any disease, disorder, condition, or complication associated with a disease, disorder, or condition, in which administration of stem cells, progenitor cells or endothelial cells is desirable. In certain embodiments, the subject is in need of treatment for a disease selected from the group consisting of cancer, peripheral vascular insufficiency, stroke, cardiovascular injury, lung injury, liver injury and kidney injury.

In certain embodiments, the one or more harvest cells can be used for other purposes. For example, the harvested stem cells can be differentiated into a T cell or NK cell, which can be further processed for allogenic cell therapy.

Dosage and Administration

The TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, can, for example, be administered as an active ingredient of a pharmaceutical composition in association with a pharmaceutical carrier or diluent. The TPO mimetics can be administered by oral, pulmonary, parental (intramuscular, intraperitoneal, intravenous (IV) or subcutaneous injection), inhalation (via a fine powder formulation), transdermal, nasal, vaginal, rectal, or sublingual routes of administration can be formulated in dosage forms appropriate for each rout of administration. See, e.g., International Publication Nos. WO1993/25221 (Bernstein et al.) and

WO1994/17784 (Pitt et al.), the relevant content of which is incorporated herein by reference.

Solid dosage forms for oral administration include capsules, tablets, pills, powders, and granules. In such solid dosage forms, the active peptide compound is admixed with at least one pharmaceutically acceptable carrier such as sucrose, lactose, or starch. Such dosage forms can also comprise, as is normal practice, additional substances other than inert diluents, e.g., lubricating agents such as magnesium stearate. In the case of capsules, tablets, and pills, the dosage forms can also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

Preparations for parental administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through bacteria retaining filter, by incorporating sterilizing agents into the compositions, by irradiating the compositions, or by heating the compositions. They can also be manufactured using sterile water, or some other sterile injectable medium immediately before use.

Administration of the TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, is typically intramuscular, subcutaneous, or intravenous. However other modes of administration such as cutaneous, intradermal or nasal can be envisaged as well. Intramuscular administration of the TPO mimetic can be achieved by using a needle to inject a suspension of the TPO mimetic composition. An alternative is the use of a needleless injection device to administer the composition (using, e.g., Bioj ector′) or a freeze-dried powder of the TPO mimetic composition.

For intravenous, cutaneous or subcutaneous injection, or injection at the site of affliction, the TPO mimetic composition such as a composition comprising a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, can be in the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation can also be employed.

Compositions for rectal or vaginal administration are preferably suppositories which can contain, in addition to the active TPO mimetic, excipients such as cocoa butter or a suppository wax. Compositions for nasal or sublingual administration are also prepared with standard excipients well known in the art.

The pharmaceutically acceptable compositions containing the TPO mimetic are administered to a subject, giving rise to an increase in stem cell and/or progenitor cell production. An amount of a TPO mimetic to increase production of at least one cell selected from the group consisting of a stem cell, a progenitor cell and an endothelial cell in the subject is defined to be an “effective dose” or an “effective amount” of the TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4. The effective amount of the TPO mimetic will depend on, e.g., the state of the subject (e.g., disease and severity of disease being treated), the physical characteristics of the subject (e.g., height, weight, etc.). The actual amount administered and rate and time-course of administration can be determined by one skilled in the art in view of the present disclosure.

In certain embodiments, the subject is administered a single dose of the effective amount of the TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4. In other embodiments, the subject is administered more than one dose of the effective amount of the TPO mimetic.

Prescription of treatment, e.g., decisions on dosage etc., is within the responsibility of general practitioners and other medical doctors, or in a veterinary context a veterinarian, and typically takes account of the disorder to be treated, the condition of the individual patient, the site of delivery, the method of administration and other factors known to practitioners. Examples of the techniques and protocols mentioned above can be found in Remington's Pharmaceutical

Sciences, 16th edition, Osol, A. ed., 1980.

Following production of the TPO mimetic, such as a TPO mimetic comprising the amino acid sequence of SEQ ID NO: 1, preferably RWJ-800088 having a structure of formula (I), or romiplostim comprising the amino acid sequence of SEQ ID NO: 3, preferably having the amino acid sequence of SEQ ID NO: 4, and optional formulation of the TPO mimetic into compositions, the compositions can be administered to an individual, particularly human or other primate. Administration can be to humans, or another mammal, e.g., mouse, rat, hamster, guinea pig, rabbit, sheep, goat, pig, horse, cow, donkey, monkey, dog or cat. Delivery to a non-human mammal need not be for a therapeutic purpose, but can be for use in an experimental context.

The TPO mimetic compositions of the application can be administered alone or in combination with other treatments (e.g., a mobilizing agent), either simultaneously or sequentially dependent upon the condition to be treated.

The TPO mimetic compositions of the application can, if desired, be presented in a kit, pack or dispenser, which can contain one or more unit dosage forms containing the active ingredient. The kit, for example, can comprise metal or plastic foil, such as a blister pack. The kit, pack, or dispenser can be accompanied by instructions for administration. The device included in the kit can be, for example, a container, a delivery vehicle, or an administration device.

### Examples

### Example 1. Effect of a TPOm on cell populations in the bone marrow of healthy mice

In this study, normal male C57BL/6 mice were subcutaneously injected with RWJ-800088 (TPOm) at 300 μg/kg (Day 0). At days 3, 7, 10 and 14, the mice were sacrificed to collect the bone marrow and blood samples.

Bone Marrow Cell Collection

Tibias were flushed and digested with 1 mg/mL collagenase IV (Gibco, 17104019) and 2 mg/mL dispase (Gibco, 17105041) in Hank's balanced salt solution (HB SS) (Gibco, 24020117) for a total of 30 minutes at 37° C. with an inversion at 15 minutes. After, the cell pellet was resuspended in 1X ACK lysing buffer (Lonza, 10-548E) to lyse red blood cells. The suspension was filtered through 70 μm nylon mesh and collected for further analysis.

The bone marrow was further processed to isolate single cell populations. Then, the cells from the bone marrow and blood were subjected to flow cytometry to identify various population, including endothelial cells (EC), EPC, arterial EC, sinusoidal EC, lineage-, c-kit+, Sca-1+(LSK), megakaryocyte progenitors, short-term hematopoietic stem cell (ST-HSC), long-term HSC (LT-HSC), mesenchymal stern cell (MSC), LepR+c-mpl+MSC. The portion of blood sample was also subjected to clonogenic assay to validate the EPC population.

Peripheral Blood Cell Collection

Blood was drawn through cardiac puncture, typically a final volume of 700 μL. Blood was collected sterilely with a heparin-coated syringe and aspirated into a tube containing heparin to prevent clotting. Blood was further diluted with endothelial cell culture medium (MEMa, 10%

FBS, 0.5% Pen Strep, and 0.05 mM 2-mercaptoethanol) to reach 2 mL final volume. Blood was separated by Histopaque-1083 density gradient centrifugation. Buffy coat was collected containing peripheral blood mononuclear cells for the analysis.

Flow Cytometry

The cells isolated from the bone marrow and peripheral blood were incubated with

Live/Dead Zombie dye (Biolegend) in phosphate-buffered saline (PBS) at room temperature for 15 minutes. After, primary antibodies were diluted in 2% fetal bovine serum (FBS)-PBS solution and incubated with cells on ice for 30 minutes. Cells were washed with 2% FBS-PBS solution and incubated with secondary antibodies for 30 minutes. Cells were washed again and acquired on Cytek Aurora spectral cytometer. Analysis was done through FlowJo (Tree Star) software.

The list of the antibodies is indicated in Tables 1 and 2.

TABLE 1
Hematopoietic flow cytometry panels

| Antibody | Fluorophore | Target Population | Notes |
|---|---|---|---|
| Lineage | PerCP-Cy5.5 | (Lin neg) ECs, HSPCs, | |
| cocktail | | SCs | |
| Sca-1 | FITC | HSPCs, AECs | |
| c-kit | APC | HSPCs, MkP | |
| CD34 | BV421 | LT-, ST-HSCs; ECFCs | |
| CD41 | AF700 | MkP, platelets | |
| c-mpl | AF647 | platelets, MkP | |
| CD150 | BV711 | LT-HSCs, MkP | |
| CD48 | APC-Cy7 | LT-HSCs | |
| Live dead | Zombie NIR | live cells | Stain in PBS alone |

TABLE 2
Endothelial/Stromal flow cytometry panels

| Antibody | Fluorophore | Target Population | Notes |
|---|---|---|---|
| CD45, | PerCP-Cy5.5 | Mature immune | |
| TER-119 | | cells | |
| CD31 | BV605 | ECs | |
| CD144 | PE-Cy7 | ECs | |
| Sca-1 | AF700 | AECs | |
| CD62P | FITC | SECs | |
| CD51 | PE | SCs | |
| CD140a | PE-Cy5 | SCs | |
| c-mpl | AF647 | Subset of SCs | |
| CD34 | BV421 | EPCs | |
| VEGFR2 | Unconjugated | All ECs | Secondary - AF594 |
| Live dead | Zombie NIR | live cells | Stain in PBS alone |

Results

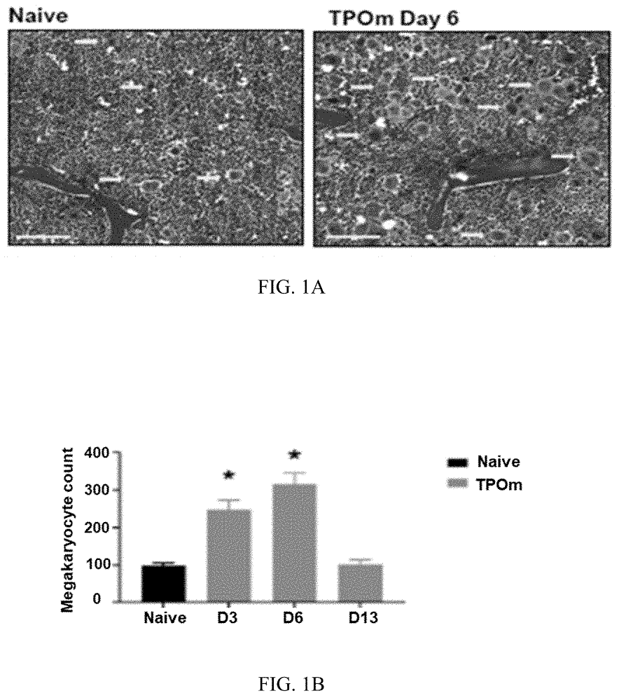

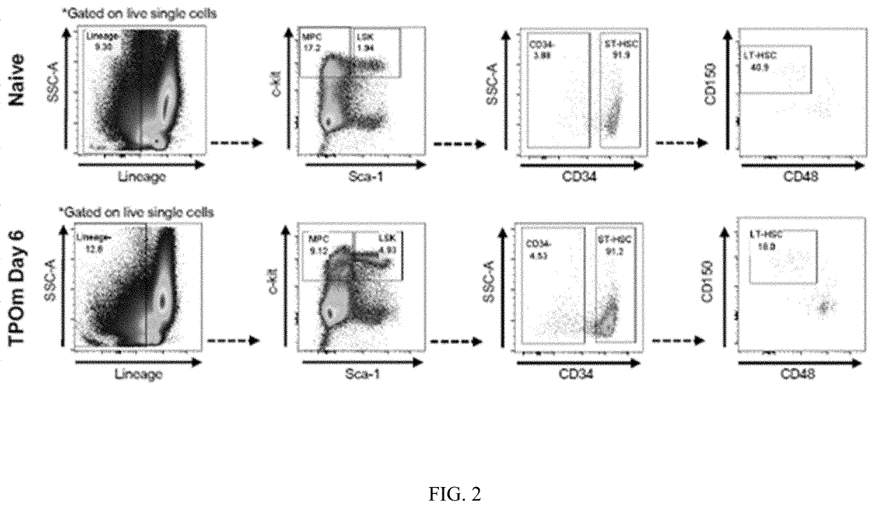

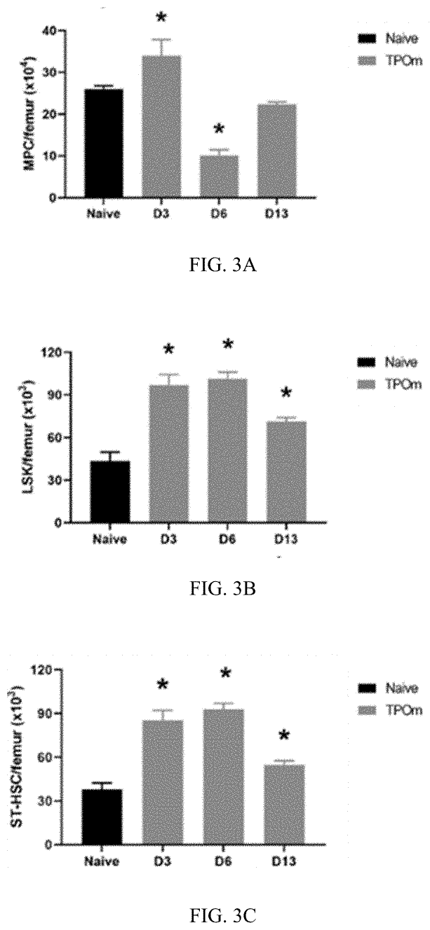

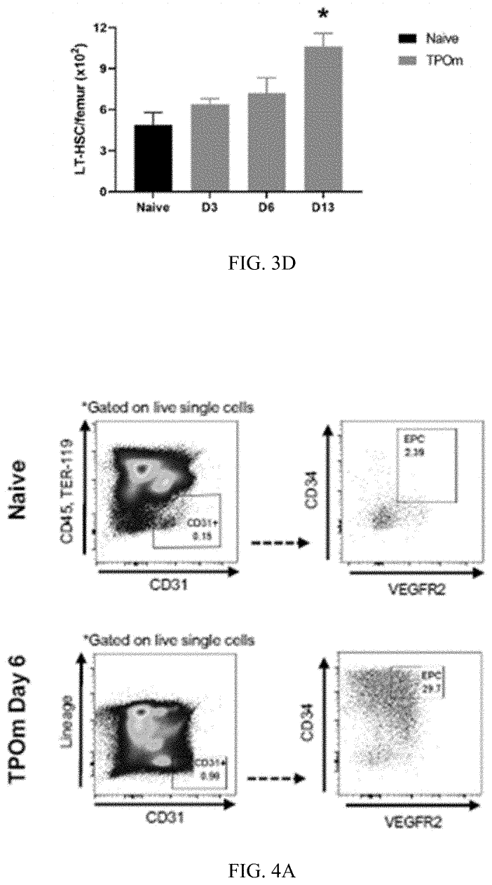

Histology with H&E staining of the sternal marrow indicated significant increases in megakaryocytes (Mks) over time in RWJ-800088 treated animals relative to naive animals (FIGS. 1A-B). This increase in marrow Mks was observed on days 3 and 6, however, the number of Mks dropped down to baseline 13 days after RWJ-800088 treatment (FIG. 1B). Further, hematopoietic stem and progenitor cell (HSPC) populations in the femoral marrow were examined by flow cytometry (FIG. 2). Myeloid progenitor cells (MPCs) indicated by lineage-, c-kit+showed significant increases on day 3 after RWJ-800088 treatment relative to naive. This was followed by a significant decrease on day 6 after RWJ-800088 treatment with a return to baseline by day 13 (FIG. 3A). The lineage-, c-kit+, Sca-1+(LSK) and the LSK, CD34+short-term (ST-) HSC populations followed a similar trend and were significantly increased after

RWJ-800088 treatment for 13 days (FIGS. 3B-3C). Finally, the LSK, CD34-, CD48-, CD150+ population which labeled the long-term (LT-) HSCs was only significantly increased by day 13 after RWJ-800088 treatment (FIG. 3D).

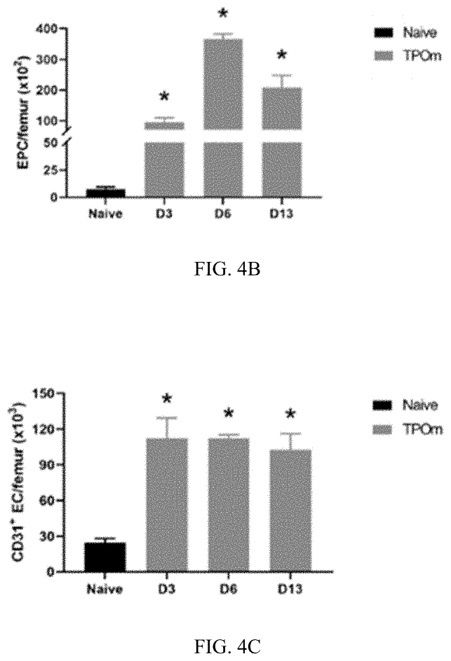

Endothelial progenitor (EPC) and CD31+endothelial cells (EC) in the femoral marrow of the mice were also examined by flow cytometry (FIG. 4A). The EPC population began increasing on day 3 with a peak on day 6, while the EC population increased beginning on day 3 and was sustained through day 13 (FIGS. 4B-4C). Results are summarized in Table 3. These results indicate that RWJ-800088 can effectively increase Mks, various hematopoietic cells, and endothelial cells in the bone marrow of healthy mice.

TABLE 3
Days the cell populations in bone marrow and peripheral
blood had significant changes following administration
of a single dose of RWJ-800088

| | | | | | | Days with |
|---|---|---|---|---|---|---|
| | | | | | | Significant |
| | | | | | | Increase |
| | | | | | | post- RWJ- |
| | | | Cell | | | 800088 |
| | | | population/ | | | treatment |
| | | | Growth | | | (vs. Naïve p < |
| Source | Group | n | factor | Method | Markers | 0.05) |
| Bone marrow | 1 | 4 | Megakaryocytes | Histological/ | N/A | D2, D6 |
| | | | | count sternebrae | | |
| | | | Myeloid progenitor | Flow cytometry- | Lineage⁻, | D3 |
| | | | cells (MPC) | total count/ | c-kit⁺ | |
| | | | LSK | femur | Lineage⁻, | D3, D6, D13 |
| | | | | | c-kit⁺, | |
| | | | | | Sca-1⁺ | |
| | | | Short-term HSCs | | Lineage⁻, | D3, D6, D13 |
| | | | | | c-kit⁺, | |
| | | | | | Sca-1⁺, | |
| | | | | | CD34⁺ | |
| | | | Long-term HSCs | | Lineage⁻, | D13 |
| | | | | | c-kit⁺, | |
| | | | | | Sca-1⁺, | |
| | | | | | CD34⁺, | |
| | | | | | CD48⁻, | |
| | | | | | CD150⁺ | |
| | | | Endothelial | | CD45⁻, | D3, D6, D13 |
| | | | progenitor cells | | TER-119⁻, | |
| | | | (EPC) | | (Lineage⁻), | |
| | | | | | CD31⁺, | |
| | | | | | CD34⁺, | |
| | | | | | VEGFR2⁺ | |
| | | | Endothelial | | CD45⁻, | D3, D6, D13 |
| | | | cells (EC) | | TER-119⁻, | |
| | | | | | (Lineage⁻), | |
| | | | | | CD31⁺ | |
| Peripheral | 3 | 3 | Endothelial | Flow cytometry- | CD45⁻, | D10 |
| Blood | | | progenitor cells | frequency of | CD31⁺, | |
| | | | | CD45⁻ (%) | CD34⁺, | |
| | | | | | CD144⁺, | |
| | | | | | VEGFR2⁺ | |
| | | | Circulating ECs | | CD45⁻, | D10 |
| | | | | | CD31⁺, | |
| | | | | | CD144⁺, | |
| | | | | | VEGFR2⁺ | |

*n = 3,\hat{ }n = 3 on Days 2, 4, 7, 14

### Example 2. Effect of plerixafor and a TPOm on cell populations in the bone marrow of healthy mice

In this study, male C57BL/6 mice (8-10 weeks old) from Jackson Laboratory (Bar Harbor, Me.) were subcutaneously injected with RWJ-800088 (TPOm) at 300 μg/kg or PBS (vehicle). At day 10 after RWJ-800088 injection, the mice were sacrificed to harvest bone marrow and peripheral blood. Another set of groups was intraperitoneally injected with 5 mg/kg of plerixafor (also named AMD3100) from Sigma-Aldrich 1 hour before the sacrifice. The bone marrow was further processed to isolate single cell populations. Then, the cells from bone marrow and blood were subjected to flow cytometry to identify various populations. Bone Marrow Cell Collection

Tibias were flushed and digested with 1 mg/mL collagenase IV (Gibco, 17104019) and 2 mg/mL dispase (Gibco, 17105041) in Hank's balanced salt solution (HBSS; Gibco, 24020117) for a total of 30 minutes at 37° C. with an inversion at 15 minutes. After, cell pellet was resuspended in lx ACK lysing buffer (Lonza, 10-548E) to lyse red blood cells. The suspension was filtered through 100 μm nylon mesh and collected for further analysis.

Peripheral Blood Cell Collection

Blood was drawn through cardiac puncture, typically a final volume of 700 μL. Blood was collected sterilely with a heparin-coated syringe and aspirated into a tube containing heparin to prevent clotting. Blood was further diluted with endothelial cell culture medium (MEMa, 10% FBS, 0.5% Pen Strep, and 0.05 mM 2-mercaptoethanol) to reach 2 mL final volume. Blood was separated by Histopaque-1083 density gradient centrifugation. Buffy coat was collected containing peripheral blood mononuclear cells for the analysis.

Flow Cytometry

The cells isolated from the bone marrow and peripheral blood were incubated with Live/Dead Zombie dye (Biolegend) in PBS at room temperature for 15 minutes. After, primary antibodies were diluted in 2% FBS-PBS solution and incubated with cells on ice for 30 minutes.

Cells were washed with 2% FBS-PBS solution and incubated with secondary antibodies for 30 minutes. Cells were washed again and acquired on Cytek Aurora spectral cytometer. Analysis was done through FlowJo (Tree Star) software. The list of the antibodies is indicated in Table 4.

The hemopoietic stem cells (HSCs) were identified as CD45-, c-kit+, Sca-1+, CD34+ and myeloid progenitor cells (MPCs) were identified as CD45-, c-kit+, Sca-1-. Endothelial progenitor cells (EPCs) were identified as CD45-, CD31+, CD144+, VEGFR2+, CD34+in bone marrow and in the peripheral blood. Circulating EC (CECs) and bone marrow ECs (BM EC) were identified with CD45-, CD31+, CD144+, VEGFR2+, CD34-.

TABLE 4
Flow cytometry panels

| Antibody | Fluorophore | Target Population |
|---|---|---|
| CD45 | PerCP-Cy5.5 | (CD45) ECs, HSPCs, MSCs |
| Sca-1 | AF700 | HSPCs |
| c-kit | APC | HSPCs |
| CD34 | BV421 | LT-, ST-HSCs; ECFCs |
| Flk1/VEGFR2 | AF594 | EC |
| VEGFR3 | NL637 | LECs |
| CD31 | FITC | ECs |
| VE-Cadherin | PE-Cy7 | ECs |
| CD51 | PE | MSCs |
| CD140a | BV605 | MSCs |
| Live dead | Zombie NIR | live cells |

Results

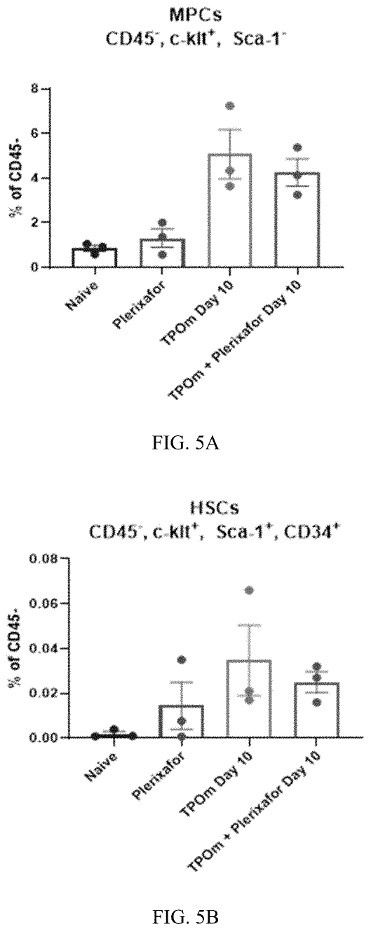

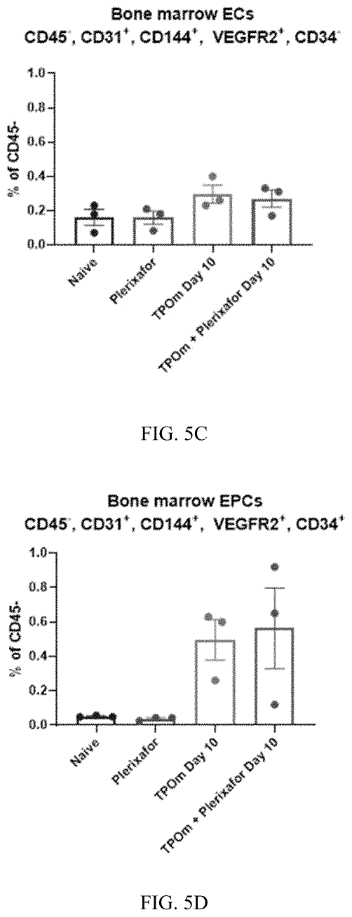

Ten days after RWJ-800088 treatment, there were increases in the MPC and HSC populations in the bone marrow compartment (FIGS. 5A-5B). These populations are known to express the thrombopoietin receptor, c-Mpl, thus making them direct targets of RWJ-800088. There was also a significant increase in EPC populations in the bone marrow 10 days after RWJ-800088 treatment (FIG. 5D), which can have the capacity to differentiate into mature ECs.

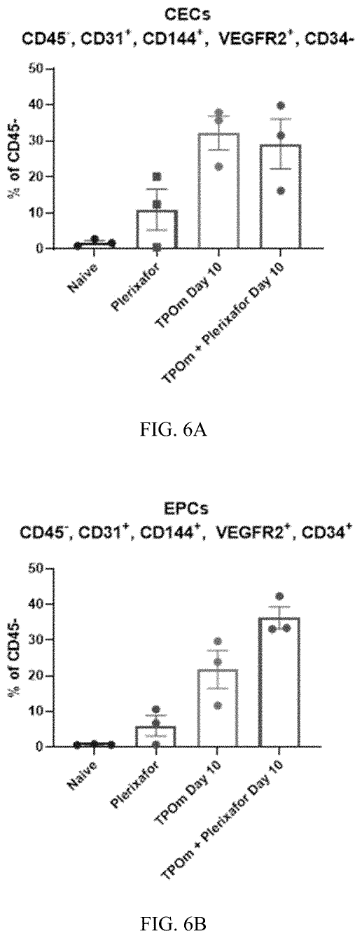

Most circulating EPCs are believed to be BM-derived (Yang et. al., 2011) and express CXCR4. Thus, it was hypothesized that plerixafor would mobilize the BM EPCs into circulation. FIGS. 6A and 6B show an increase in circulating ECs (CECs) as well as mobilized EPCs after RWJ-800088 and plerixafor treatment.

### Example 3. Effect of a TPOm on cell populations in the bone marrow of healthy mice

In this study, normal male C57BL/6 mice are subcutaneously injected with romiplostim (SEQ ID NO: 3) (TPOm) at a desired amount or amounts, such as, for example, 300 μg/kg (Day 0). At various timepoints, the mice are sacrificed to collect the bone marrow and blood samples. Bone Marrow Cell Collection

Tibias are flushed and digested with collagenase IV (Gibco, 17104019) and dispase (Gibco, 17105041) in Hank's balanced salt solution (HBSS) (Gibco, 24020117). After, the cell pellet is resuspended in lysing buffer (Lonza, 10-548E) to lyse red blood cells. The suspension is filtered through a nylon mesh and collected for further analysis.

The bone marrow is further processed to isolate single cell populations. Then, the cells from the bone marrow and blood are subjected to flow cytometry to identify various population, including endothelial cells (EC), EPC, arterial EC, sinusoidal EC, lineage-, c-kit+, Sca-1+(LSK), megakaryocyte progenitors, short-term hematopoietic stem cell (ST-HSC), long-term HSC (LT-HSC), mesenchymal stem cell (MSC), LepR+c-mpl+MSC. The portion of blood sample is also subjected to clonogenic assay to validate the EPC population.

Peripheral Blood Cell Collection

Blood is drawn through cardiac puncture, typically a final volume of 700 μL. Blood is collected sterilely with a heparin-coated syringe and aspirated into a tube containing heparin to prevent clotting. Blood is further diluted with endothelial cell culture medium (MEMa, 10% FBS, 0.5% Pen Strep, and 0.05 mM 2-mercaptoethanol) to reach a desired final volume. Blood is separated by Histopaque-1083 density gradient centrifugation. Buffy coat is collected containing peripheral blood mononuclear cells for the analysis.

### Flow Cytometry

The cells isolated from the bone marrow and peripheral blood are incubated with Live/Dead Zombie dye (Biolegend) in phosphate-buffered saline (PBS) at room temperature.

After, primary antibodies are diluted in 2% fetal bovine serum (FBS)-PBS solution and incubated with cells on ice for 30 minutes. Cells are washed with 2% FBS-PBS solution and incubated with secondary antibodies. Cells are washed again and acquired on Cytek Aurora spectral cytometer. Analysis is done through FlowJo (Tree Star) software. The list of the antibodies is indicated in Tables 5 and 6.

TABLE 5
Hematopoietic flow cytometry panels

| Antibody | Fluorophore | Target Population | Notes |
|---|---|---|---|
| Lineage | PerCP-Cy5.5 | (Lin neg) ECs, HSPCs, | |
| cocktail | | SCs | |
| Sca-1 | FITC | HSPCs, AECs | |
| c-kit | APC | HSPCs, MkP | |
| CD34 | BV421 | LT-, ST-HSCs; ECFCs | |
| CD41 | AF700 | MkP, platelets | |
| c-mpl | AF647 | platelets, MkP | |
| CD150 | BV711 | LT-HSCs, MkP | |
| CD48 | APC-Cy7 | LT-HSCs | |
| Live dead | Zombie NIR | live cells | Stain in PBS alone |

TABLE 6
Endothelial/Stromal flow cytometry panels

| Antibody | Fluorophore | Target Population | Notes |
|---|---|---|---|
| CD45, | PerCP-Cy5.5 | Mature immune | |
| TER-119 | | cells | |
| CD31 | BV605 | ECs | |
| CD144 | PE-Cy7 | ECs | |
| Sca-1 | AF700 | AECs | |
| CD62P | FITC | SECs | |
| CD51 | PE | SCs | |
| CD140a | PE-Cy5 | SCs | |
| c-mpl | AF647 | Subset of SCs | |
| CD34 | BV421 | EPCs | |
| VEGFR2 | Unconjugated | All ECs | Secondary - AF594 |
| Live dead | Zombie NIR | live cells | Stain in PBS alone |

Results

Histology with H&E staining of the sternal marrow is performed to analyze megakaryocytes (Mks) over time in romiplostim treated animals relative to naive animals. Further, hematopoietic stem and progenitor cell (HSPC) populations in the femoral marrow are examined by flow cytometry. Endothelial progenitor (EPC) and CD31+endothelial cells (EC) in the femoral marrow of the mice are also examined by flow cytometry.

### Example 4. Effect of Plerixafor and TPOm on Cell Populations in the Bone Marrow of Healthy Mice

In this study, male C57BL/6 mice (8-10 weeks old) from Jackson Laboratory (Bar Harbor, Me.) are subcutaneously injected with romiplostim (SEQ ID NO: 3) (TPOm) at a desired amount, such as, for example, 300 μg/kg or PBS (vehicle). At a desired day, such as day 10, after romiplostim injection, the mice are sacrificed to harvest bone marrow and peripheral blood. Another set of groups is intraperitoneally injected with a desired amount of plerixafor, such as, for example, 5 mg/kg of plerixafor (also named AMD3100) from Sigma-Aldrich, at a desired timepoint, such as, for example, 1 hour before the sacrifice. The bone marrow is further processed to isolate single cell populations. Then, the cells from bone marrow and blood are subjected to flow cytometry to identify various populations.

### Bone Marrow Cell Collection

Tibias are flushed and digested with collagenase IV (Gibco, 17104019) and dispase (Gibco, 17105041) in Hank's balanced salt solution (HBSS; Gibco, 24020117). After, cell pellet is resuspended in lysing buffer (Lonza, 10-548E) to lyse red blood cells. The suspension is filtered through a nylon mesh and collected for further analysis.

### Peripheral Blood Cell Collection

Blood is drawn through cardiac puncture, typically a final volume of 700 μL. Blood is collected sterilely with a heparin-coated syringe and aspirated into a tube containing heparin to prevent clotting. Blood is further diluted with endothelial cell culture medium (MEMa, 10%

FBS, 0.5% Pen Strep, and 0.05 mM 2-mercaptoethanol) to reach a desired final volume, such as, for example, 2 mL final volume. Blood is separated by Histopaque-1083 density gradient centrifugation. Buffy coat is collected containing peripheral blood mononuclear cells for the analysis.

### Flow Cytometry

The cells isolated from the bone marrow and peripheral blood are incubated with Live/Dead Zombie dye (Biolegend) in PBS at room temperature. After, primary antibodies are diluted in 2% FBS-PBS solution and incubated with cells on ice. Cells are washed with 2% FBS-PBS solution and incubated with secondary antibodies. Cells are washed again and acquired on Cytek Aurora spectral cytometer. Analysis is done through FlowJo (Tree Star) software. The list of the antibodies is indicated in Table 4.

The hemopoietic stem cells (HSCs) are identified as CD45-, c-kit+, Sca-1+, CD34+ and myeloid progenitor cells (MPCs) were identified as CD45-, c-kit+, Sca-1-. Endothelial progenitor cells (EPCs) were identified as CD45-, CD31+, CD144+, VEGFR2+, CD34+in bone marrow and in the peripheral blood. Circulating EC (CECs) and bone marrow ECs (BM EC) were identified with CD45-, CD31+, CD144+, VEGFR2+, CD34-.

TABLE 4
Flow cytometry panels

| Antibody | Fluorophore | Target Population |
|---|---|---|
| CD45 | PerCP-Cy5.5 | (CD45) ECs, HSPCs, MSCs |
| Sca-1 | AF700 | HSPCs |
| c-kit | APC | HSPCs |
| CD34 | BV421 | LT-, ST-HSCs; ECFCs |
| Flk1/VEGFR2 | AF594 | EC |
| VEGFR3 | NL637 | LECs |
| CD31 | FITC | ECs |
| VE-Cadherin | PE-Cy7 | ECs |
| CD51 | PE | MSCs |
| CD140a | BV605 | MSCs |
| Live dead | Zombie NIR | live cells |

Results

After romiplostim treatment, MPC and HSC populations in the bone marrow compartment are analyzed. These populations are known to express the thrombopoietin receptor, c-Mpl, thus making them direct targets of romiplostim. EPC populations in the bone marrow are also analyzed after romiplostim treatment. EPC populations can have the capacity to differentiate into mature EC.

Most circulating EPCs are believed to be BM-derived (Yang et. al., 2011) and express

CXCR4. Plerixafor may mobilize the BM EPCs into circulation. Any potential ECs (CECs) as well as mobilized EPCs, if present, are the subject of analysis after romiplostim and plerixafor treatment.

It will be appreciated by those skilled in the art that changes could be made to the embodiments described above without departing from the broad inventive concept thereof. It is understood, therefore, that this application is not limited to the particular embodiments disclosed, but it is intended to cover modifications within the spirit and scope of the present application as defined by the present description.

It is claimed:

1. A method of obtaining at least one cell selected from the group consisting of a hematopoietic progenitor cell, a myeloid progenitor cell, an endothelial progenitor cell and an endothelial cell from a non-irradiated subject, comprising:
(a) administering an effective amount of a thrombopoietin (TPO) mimetic to the subject to increase the at least one cell in the subject;
(b) harvesting the at least one cell from the subject 7 to 21 days after the administering; and
(c) isolating the at least one cell;
wherein the TPO mimetic comprises the amino acid sequence of SEQ ID NO: 1, preferably wherein the TPO mimetic has the following structure of formula (I), or a pharmaceutically acceptable salt or ester thereof: 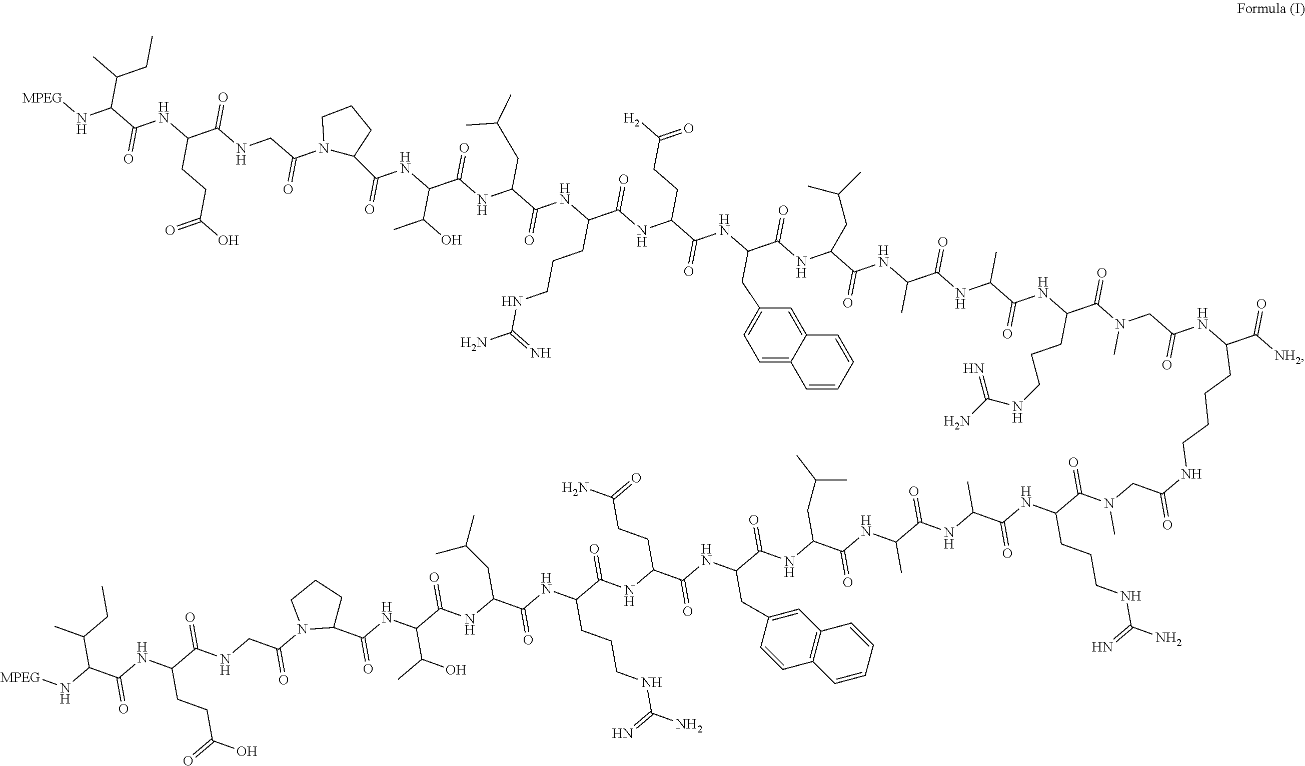
wherein MPEG represents methoxypolyethyleneglyco120000; or has the amino acid sequence of SEQ ID NO: 3 or SEQ ID NO: 4.

2. A method of providing at least one cell selected from the group consisting of a hematopoietic progenitor cell, a myeloid progenitor cell, an endothelial progenitor cell and an endothelial cell to a subject, comprising:
(a) administering an effective amount of a thrombopoietin (TPO) mimetic to the subject to increase production of the at least one cell in the subject;
(b) harvesting the at least one cell from the subject 7-21 days after the administering;
(c) isolating the at least one cell; and
(d) administering the isolated at least one cell to the subject,
wherein the TPO mimetic comprises the amino acid sequence of SEQ ID NO: 1, preferably wherein the TPO mimetic has the following structure of formula (I), or a pharmaceutically acceptable salt or ester thereof: 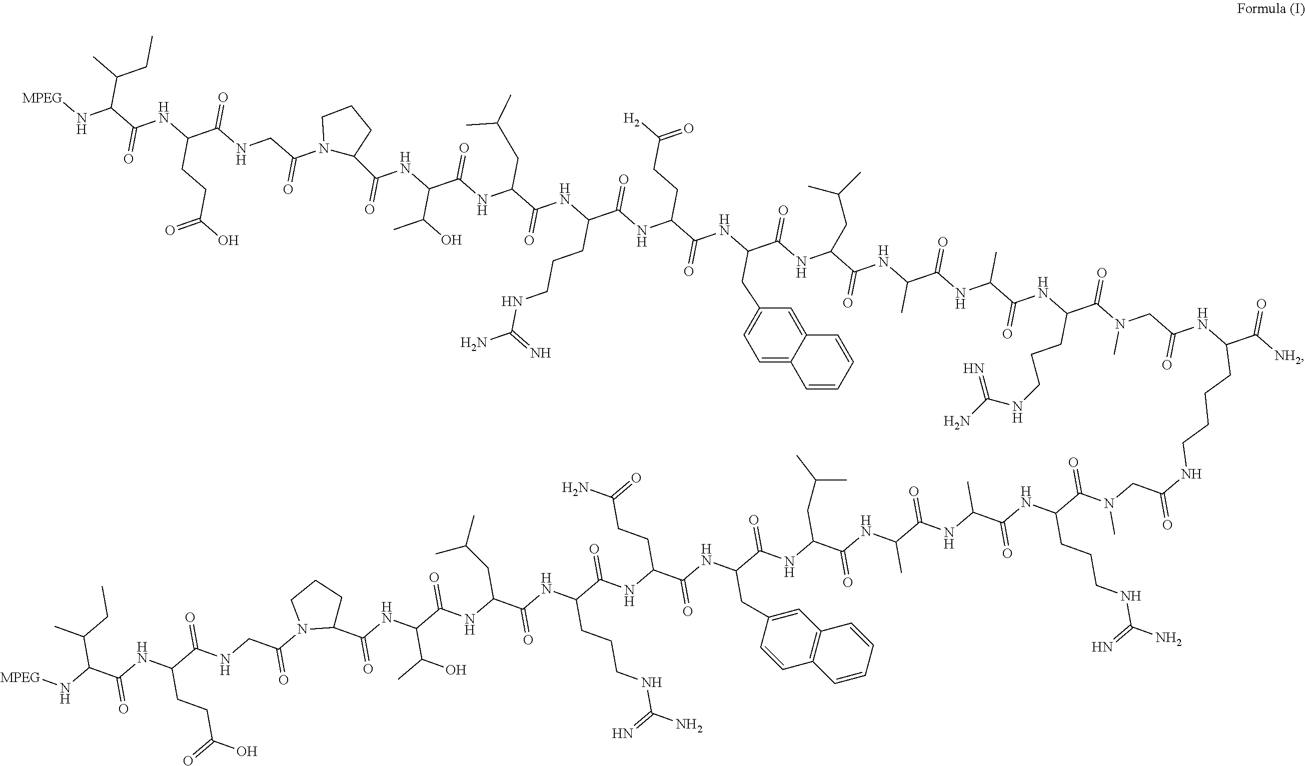
wherein MPEG represents methoxypolyethyleneglyco120000; or has the amino acid sequence of SEQ ID NO: 3 or SEQ ID NO: 4.

3. The method of claim 1 or claim 2, wherein the TPO mimetic has the following structure of formula (I), or a pharmaceutically acceptable salt or ester thereof: 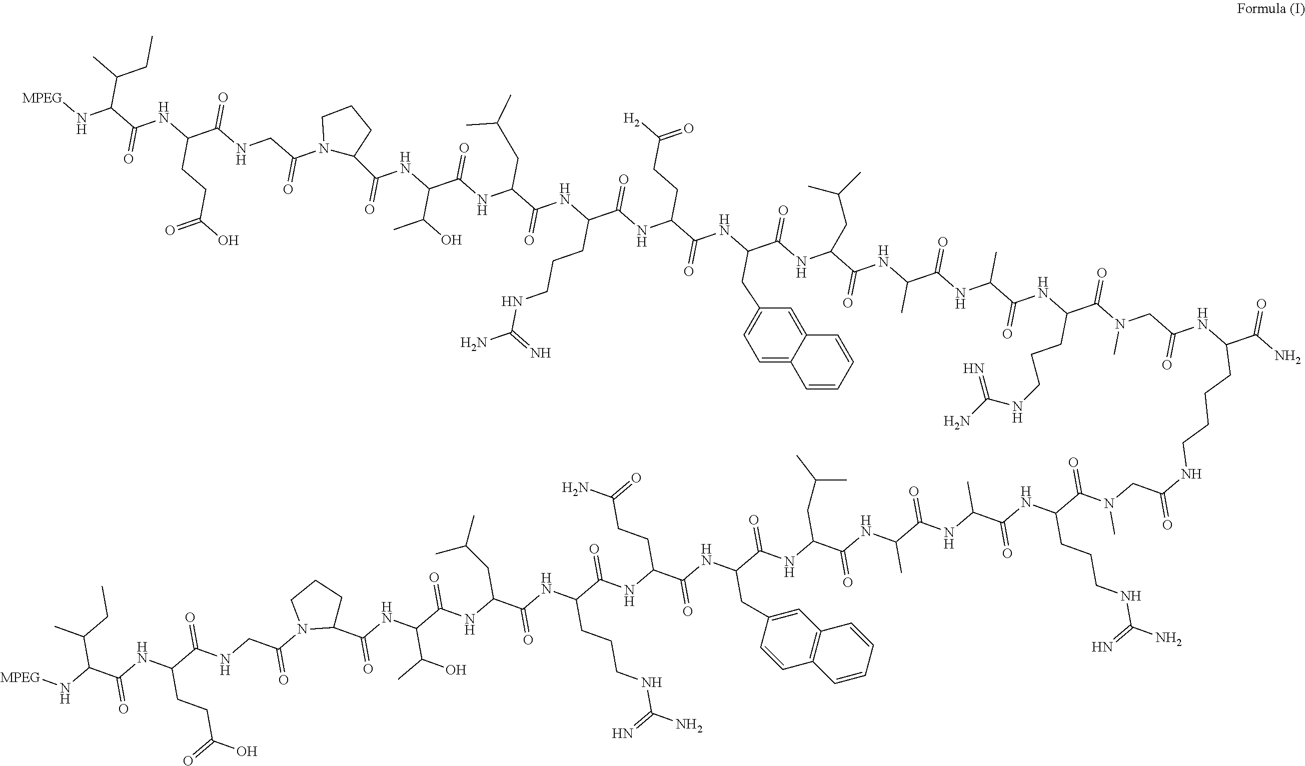
wherein MPEG represents methoxypolyethyleneglyco120000.

4. The method of any one of claims 1-3, further comprising administering to the subject a mobilizing agent prior to harvesting the at least one cell.

5. The method of claim 4, wherein the mobilizing agent is administered after the TPO mimetic is administered

6. The method of claim 4 or 5, wherein the mobilizing agent is plerixafor or an FLT-3 ligand.

7. A method of treating a disease or disorder in a subject in need thereof, comprising administering to the subject an effective amount of at least one cell selected from the group consisting of a hematopoietic progenitor cell, a myeloid progenitor cell, an endothelial progenitor cell and an endothelial cell to a subject, wherein the at least one cell is obtained by a method comprising:
(a) administering an effective amount of a thrombopoietin (TPO) mimetic to the subject to increase production of the at least one cell in the subject;
(b) harvesting the at least one cell; and
(c) isolating the at least one cell; wherein the TPO mimetic comprises the amino acid sequence of SEQ ID NO: 1, preferably wherein the TPO mimetic has the following structure of formula (I), or a pharmaceutically acceptable salt or ester thereof: 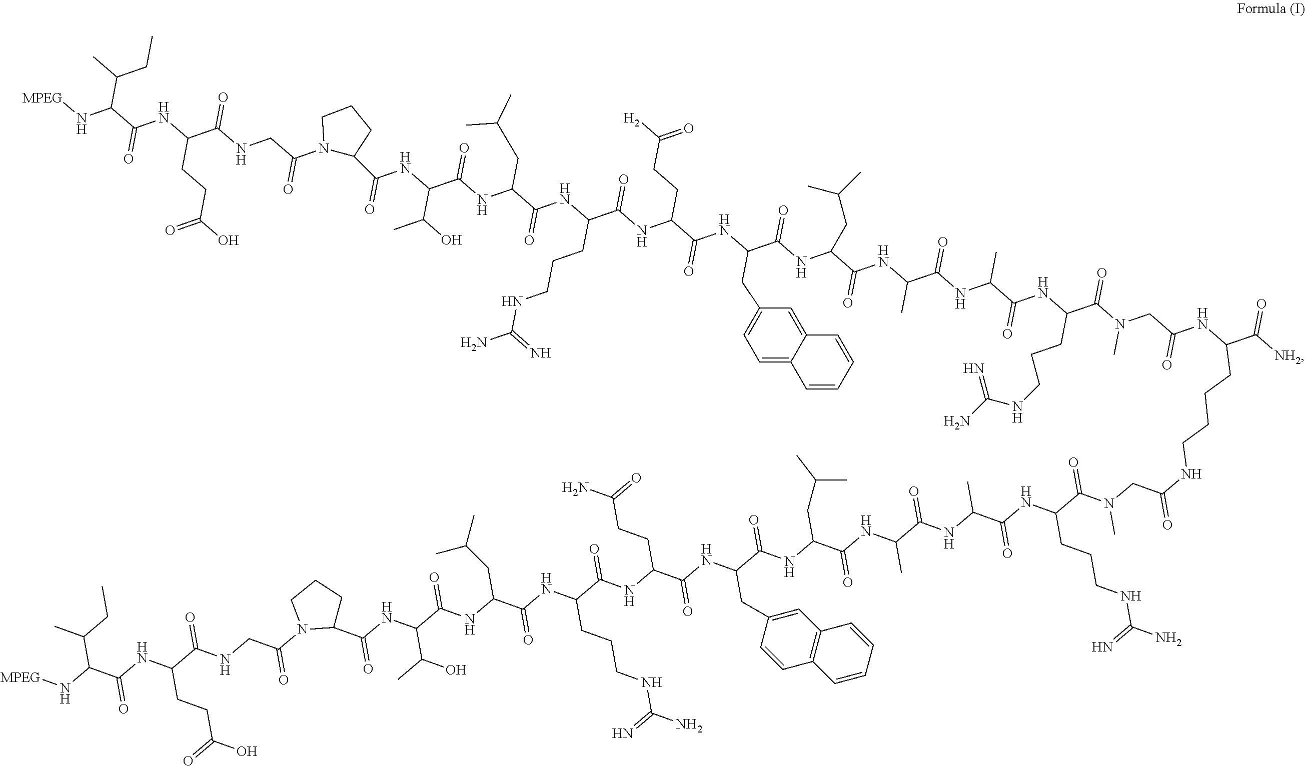
wherein MPEG represents methoxypolyethyleneglyco120000; or has the amino acid sequence of SEQ ID NO: 3 or SEQ ID NO: 4.

8. The method of claim 7, wherein the TPO mimetic has the following structure of formula (I), or a pharmaceutically acceptable salt or ester thereof: 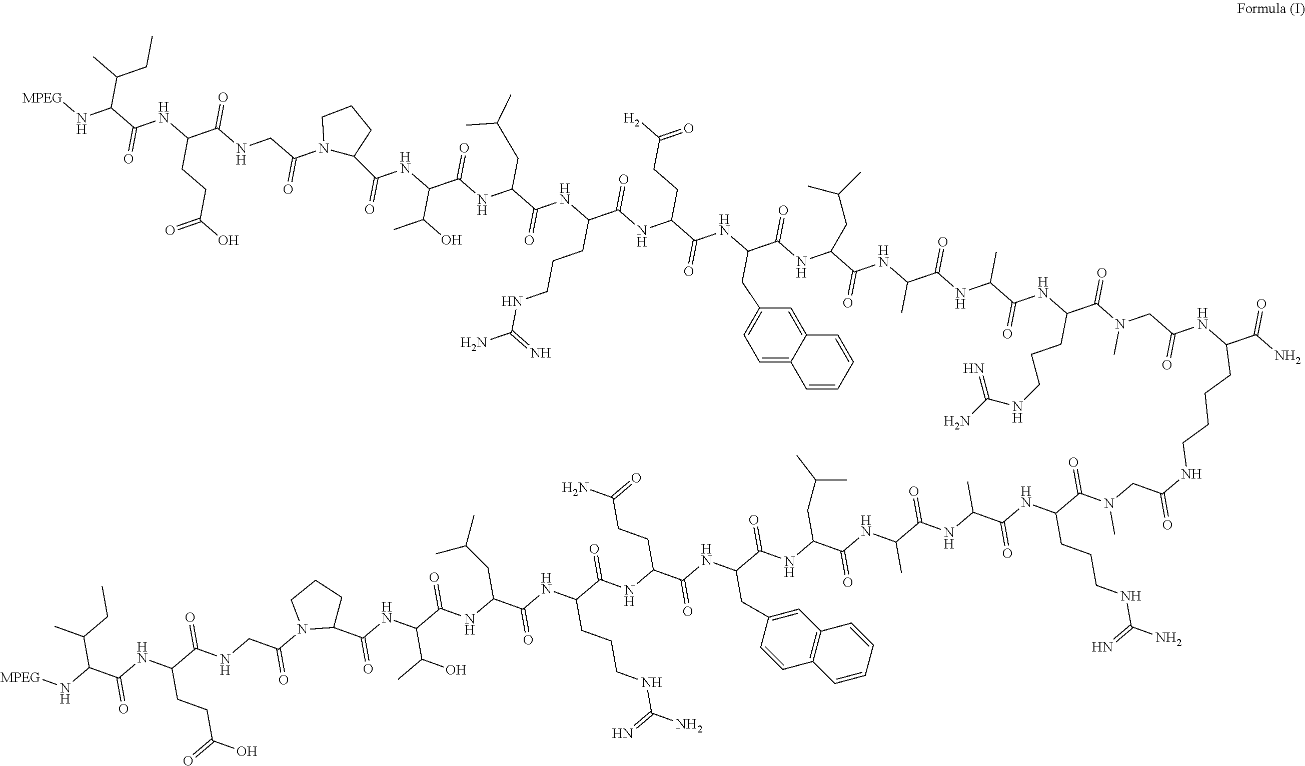
wherein MPEG represents methoxypolyethyleneglyco120000.

9. The method of claim 7 or claim 8, further comprising administering plerixafor to the subject 7-20 days after administering the TPO mimetic.

10. The method of any one of claims 1-9, further comprising treating the subject with a bone marrow ablative treatment after harvesting the at least one cell.

11. The method of any one of claims 1-10, wherein the subject is in need of treatment for a disease selected from the group consisting of cancer, peripheral vascular insufficiency, stroke, cardiovascular injury, lung injury, liver injury and kidney injury.

12. The method of any one of claims 1-11, wherein the subject is a human.

13. The method of any one of claims 1-12, wherein the number of the at least one cell is increased within the subject's bone marrow.

14. The method of any one of claims 1-13, wherein the number of the at least one cell is increased within the subject's peripheral circulation.

15. The method of any one of claims 1-14, wherein the at least one cell is harvested from the subject's peripheral circulation.

16. The method of any one of claims 1-15, wherein the at least one cell is cryopreserved after harvesting.

17. The method of claim 16, wherein the cryopreserved at least one cell is thawed and determined to be viable prior to administering the cells to the subject.

18. The method of claim 17, wherein the at least one cell is administered to the subject when the subject is in need of such cell administration.

19. The method of any one of the preceding claims, wherein the TPO mimetic is administered to the subject by intravenous, intramuscular, intracutaneous, or subcutaneous injection.

20. The method of any one of the preceding claims, wherein the subject is administered a single dose of the effective amount of the TPO mimetic.

21. The method of any one of claims 1-19, wherein the subject is administered more than one dose of the effective amount of the TPO mimetic.


SEQUENCE LISTING (4 sequences)

SEQ ID NO: 1 - protein, 14 aa, synthetic construct
  REGION 1..14: note = Synthetic Sequence
  SITE 9: note = X - tryptophan or beta-(2-naphthyl)alanine
  SITE 14: note = X - alanine or sarcosine
IEGPTLRQXLAARX

SEQ ID NO: 2 - protein, 14 aa, synthetic construct
  REGION 1..14: note = Synthetic peptide
  SITE 9: note = X - beta -(2-naphthyl)alanine
  SITE 14: note = X - sarcosine
IEGPTLRQXLAARX

SEQ ID NO: 3 - protein, 14 aa, synthetic construct
  REGION 1..14: note = Synthetic peptide
IEGPTLRQWLAARA

SEQ ID NO: 4 - protein, 269 aa, synthetic construct [sequence omitted]